(19) United States
(12) Patent Application Publication
(10) Pub. No.: US 2024/0172965 A1
(43) Pub. Date: May 30, 2024

(54) MOTION MOTOR TEST SYSTEM

(71) Applicant: Nanyang Technological University, Singapore (SG)

(72) Inventors: Wee Kiat TAN, Singapore (SG); Wei Tech ANG, Singapore (SG); Lek Syn LIM, Singapore (SG); Shupei Phyllis LIANG, Singapore (SG); Wai Hang KWONG, Singapore (SG); Ananda Ekaputera SIDARTA, Singapore (SG)

(21) Appl. No.: 18/553,149

(22) PCT Filed: Apr. 19, 2022
(86) PCT No.: PCT/SG2022/050230

§ 371 Date: Sep. 28, 2023

(30) Foreign Application Priority Data
Apr. 20, 2021 (SG) 10202104026V

(51) Int. Cl. A61B 5/11; A61B 5/00

(52) U.S. Cl. CPC A61B 5/1118; A61B 5/1125; A61B 5/6891

(57) ABSTRACT

A motion motor test system is described. The motion motor test system comprises: a simulated key portion which is rotatable around a rotational axis; a rotational resistive load mechanically coupled to the simulated key portion; and a sensor subsystem coupled to the simulated key portion configured to measure force and/or torque applied to the simulated key portion.

### TECHNICAL FIELD

The present disclosure relates to motion motor test systems for collecting kinetic data of a subject while the subject is carrying out a motion motor dexterity test.

### BACKGROUND

Assessment of human movement performance in activities of daily living (ADL) is one of the key components in clinical and rehabilitation settings. Many clinical tests assess human movement in ADL tasks, and the instrument used can vary from a complex setup to a simple test kit.

One of the most robust and accurate methods is the use of motion capture technology for objective assessment of human movement in term of the level of impairment and function in people with movement impairment, such as stroke survivors, to track the level of recovery and determine the efficacy of treatments. This technology can capture subject posture in time-varying dimensional movement data by using infrared reflective markers, and specialized cameras. These specialized cameras can emit infrared light and capture the infrared light reflected by the markers. By using a correlation algorithm, the coordinate position and time of each marker will be known in the three-dimensional space. However, motion capture technology is a costly investment and mostly be used in the institution and hospital setting. One of the shortcomings of this technology is that it only provided kinematic data which can be insufficient to assess the human movement as a human movement do involve kinetic data. Though force plates are used to assess the force used during walking, they cannot be used to assess upper limbs movement performance. Also, the putting on of reflective markers onto the subject body create inconvenience to the patients.

Therefore, most therapists use test kits like the Action Research Arm Test (ARAT) or the Wolf Motor Function (WMFT) in a community setting. The ARAT Test is a 19-item observational measure used by therapists and other health care workers to assess upper extremity performance in terms of coordination, dexterity and functioning for stroke recovery, brain injury and multiple sclerosis patients. ARAT is categorized into four subscales which are the grasp, grip, pinch, and gross movement tasks and arranged in decreasing difficulty. Task performance is rated on a 4-point scale, ranging from 0 for no movement to 3 for normal movement. For the WMFT, it is a time-based method that had 15 tasks to assess upper extremity performance with a similar 5-point scoring method. In the ARAT and WMFT tests, the therapist will visually observe the subject body posture while doing the test. The therapist will then give a score based on his/her judgment on subject movement.

But these standardized clinical assessments have few issues. Firstly, these standardized clinical assessments tend to assess the patients' performance with a total score or estimate. This causes information loss like the variability in strength control. Secondly, these tests are insensitive in picking up subtle changes in motor performance and detecting abnormalities in patients as it depends on the therapist observation. Lastly, the tests are too heavily dependable on the therapist's judgement and visual observation which can cause a large variation in the results.

Overall, this shows that there are the following constraints with the current assessment tests: (i) Highly accurate and reliable equipment like a motion capture system is too costly to be used in community-based tests; (ii) Current clinical tests and scoring are dependent on the tester experience in executing the ARAT and WMFT test; (iii) Lack of kinetic data in small test kits which cannot be shared among different patients; and (iv) Scoring used by clinical tests is very hard to correlate with the kinematic data.

### SUMMARY

According to an aspect of the present disclosure a motion motor test system is provided. The motion motor test system comprises: a simulated key portion which is rotatable around a rotational axis; a rotational resistive load mechanically coupled to the simulated key portion; and a sensor subsystem coupled to the simulated key portion configured to measure force and/or torque applied to the simulated key portion.

The motion motor test system allows monitoring of a turning of a key task from the signals generated by the sensor subsystem.

In an embodiment, the simulated key portion comprises a key bow and the sensor subsystem comprises a load cell provided in the key bow configured to measure a pinch force applied to the key bow. This allows the pinch force to be measured by the sensor subsystem. The key bow may comprise two flat portions with the load cell provided between the two flat portions.

The motion motor test system may further comprise: an arm portion mechanically coupled to the key portion and extending radially from the rotational axis, and wherein the sensor subsystem comprises a first load cell mounted in a position offset from the rotational axis and configured to measure a force applied by the arm portion. This allows the turning force to be measured by the sensor subsystem. The first load cell may be mounted in a rotational position at which a key turning action is completed.

The motion motor test system may further comprise a second load cell mounted in a position offset from the rotational axis and configured to measure a force applied by the arm portion. The first load cell may be mounted in a rotational position at which a key turning action is completed in a first rotational direction and the second cell may be mounted in a rotational position at which a key turning action is completed in a second rotational direction opposite from the first rotational direction.

In an embodiment, the sensor subsystem comprises a torque sensor mechanically coupled to the simulated key portion and configured to measure a torque around the rotational axis applied to the simulated key portion.

The resistive load may be provided by a lock and a key. Thus, the resistive load when operating the simulated key portion corresponds to a real lock and key.

In an embodiment, the motion motor test system further comprises a data acquisition unit configured to collect signals from the sensor subsystem and generate kinetic data for the subject based on the signals from the sensor subsystem.

In an embodiment, the motion motor test system further comprises a data synchronisation unit configured to synchronise the kinetic data for the subject with kinematic data for the subject captured from a motion capture system.

### BRIEF DESCRIPTION OF THE DRAWINGS

In the following, embodiments of the present invention will be described as non-limiting examples with reference to the accompanying drawings in which:

FIG. 1A to FIG. 1C show a motion motor test system according to an embodiment of the present invention;

FIG. 2A and FIG. 2B show examples of keys, a lock cylinder and a lock panel which may be used in embodiments of the present invention;

FIG. 3A shows a simulated key portion of a motion motor test system according to an embodiment of the present invention;

FIG. 3B shows the arrangement of a load cell within the key bow portion of the simulated key portion shown in FIG. 3A;

FIG. 3C shows an example of a miniature load cell used in embodiments of the present invention;

FIG. 4A and FIG. 4B show a motion motor test system according to an embodiment of the present invention;

FIG. 5 is a block diagram showing a system for correlating kinetic data captured from a motion motor test system according to an embodiment of the present invention with kinematic data captured by a motion capture system

### DETAILED DESCRIPTION

The present disclosure provides a motion motor test system for quantifying a patient's motor mobility when performing the task of turning a key in a lock. The motion motor test system may be used as part of the Wolf Motor Motion Test. Wolf Motor Motion Test is a 15-tasks test to quantify upper extremity movement ability through timed single or multiple joint motions and functional tasks. Turning a key in a lock is part of the 15-tasks. This test used to observe whether the patient can pincer grasp and turn the key fully to the left and right while maintaining contact. However, the scoring method used by Wolf Motor Motion Test is unable to show the detail of any abnormal behavior. The motion motor test system of the present disclosure allows parameters such as torque and force to be measured while a patient is performing the task of turning a key in a lock.

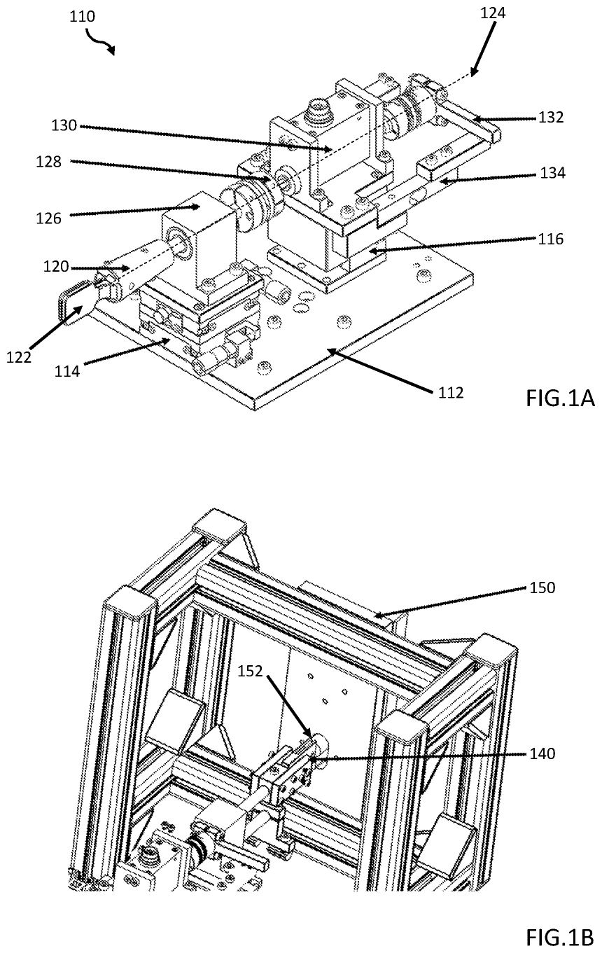

FIG. 1A to FIG. 1C show a motion motor test system according to an embodiment of the present invention. The motion motor test system comprises a rotatable assembly which is shown in FIG. 1A. The rotatable assembly is coupled to a lock panel as shown in FIG. 1B which acts as a rotational resistive load. The rotatable assembly is mounted on a frame as shown in FIG. 1C.

As shown in FIG. 1A, the rotatable assembly 110 of the motion motor test system is mounted on a mounting plate 112 and supported by a x-y adjustment block 114 and a height adjustment block 116. The rotatable assembly 110 comprises a simulated key portion 120 which has a key bow portion 122. The simulated key portion 120 is rotatable around a rotational axis 124. A bearing block 126 is mounted on the x-y adjustment block 114. The bearing block 126 allows the simulated key portion 120 and other parts of the rotatable assembly 110 to be rotated around the rotational axis 124. A coupling portion 128 couples the simulated key portion 120 to a torque sensor 130. An arm portion 132 is mounted on the rotatable assembly 110 which extends from the torque sensor 130. The arm portion 132 extends radially from the rotational axis 124. Load cells 134 are mounted on each side of the height adjustment block 116.

As shown in FIG. 1B, a coupling portion 140 of the rotatable assembly 110 grips a key 152 which is inserted into a lock cylinder of a lock panel 150. The lock panel 150 is supported by the frame of the motion motor test system.

As shown in FIG. 1C, the motion motor test system 100 comprises a frame which comprises a main frame portion 102, frame support portions 104 and a lock panel support portion 106. The frame support portions 104 form a base in contact with the floor. The main frame portion 102 extends upwards from the frame support portions 104 and supports the mounting panel 112 on which the rotatable assembly 110 is mounted. The lock panel support portion 106 is coupled to the main frame portion 102 and supports the lock panel 150 such that a key hole of the lock panel is located on the rotational axis 124 of the rotational assembly.

The main frame portion may be arranged such that the simulated key portion 120 is positioned at a height of 97 cm to simulate to the common door key height used in flats and buildings.

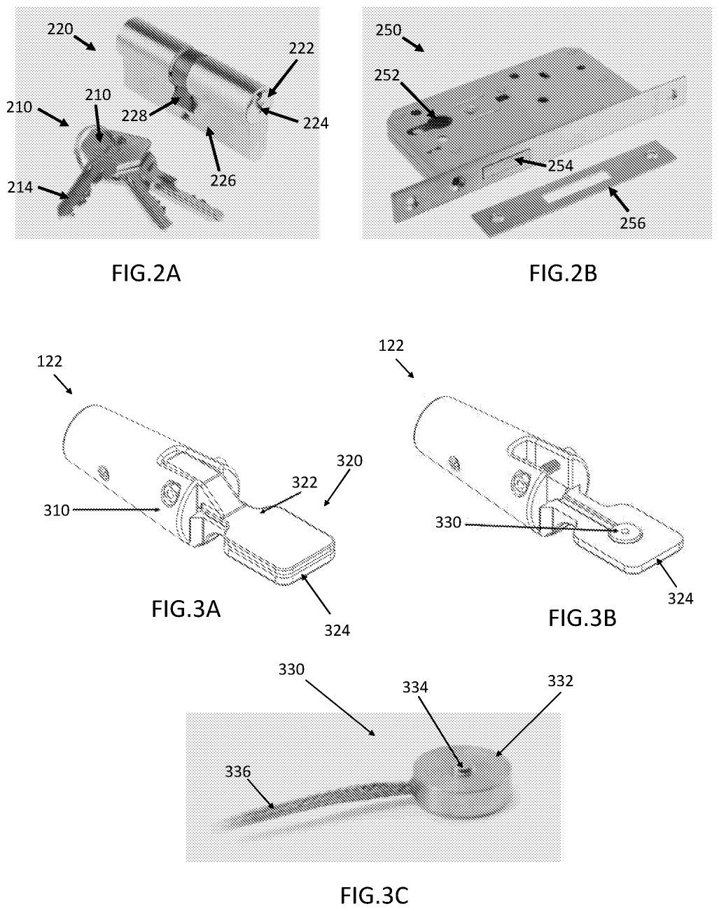

As is described in more detail below with reference to FIG. 3A to FIG. 3C, the key bow portion 122 is provided with a load cell arranged to measure a pinching force applied by a subject when manipulating the motion motor test system 100.

In use, the motion motor test system 100 allows the forces and torques applied to the simulated key portion 120 to be measured when a subject carried out an exercise of turning a key in a lock. The subject grips the key bow portion 122 of the simulated key portion 120. The load cell in the key bow portion 122 measures the pinch force applied to the key bow portion while the exercise is carried out to measure pinch force exerted by the thumb and index finger of the subject when turning the simulated key portion 120.

As the subject turns the simulated key portion 120, the rotatable assembly 110 turns around the rotational axis 124. This causes the key 152 to turn within the lock panel 150 to turn. The lock panel 150 thus acts as a rotational resistive load which provides a resistance to the turning force applied by the subject. The motion motor test system 100 thus provides an accurate simulation of the act of turning a key in a lock since the resistive force is provided by a lock itself. During the rotation of the rotatable assembly 110, the torque sensor 130 measures the torque applied by the subject. The subject may continue applying more torque even when the key already reached the end of the rotation. Therefore, the arm portion 132 is provided and when the key reaches the end of its rotation in the lock panel 150, the arm portion 132 contacts a load cell 134 mounted on the height adjustment block 116. It is noted that a load cell 134 is mounted on each side of the height adjustment block 116 so the arm portion 132 will contact one of the load cells when the key is fully rotated in the clockwise direction and will contact the other load cell when fully rotated in the anti-clockwise direction.

The load cell mounted in the key bow portion 122, the torque sensor 130, and the load cells 134 mounted on the height adjustment block 116 may be considered as a sensor sub-system which generates kinetic data during the test of turning the key in the lock.

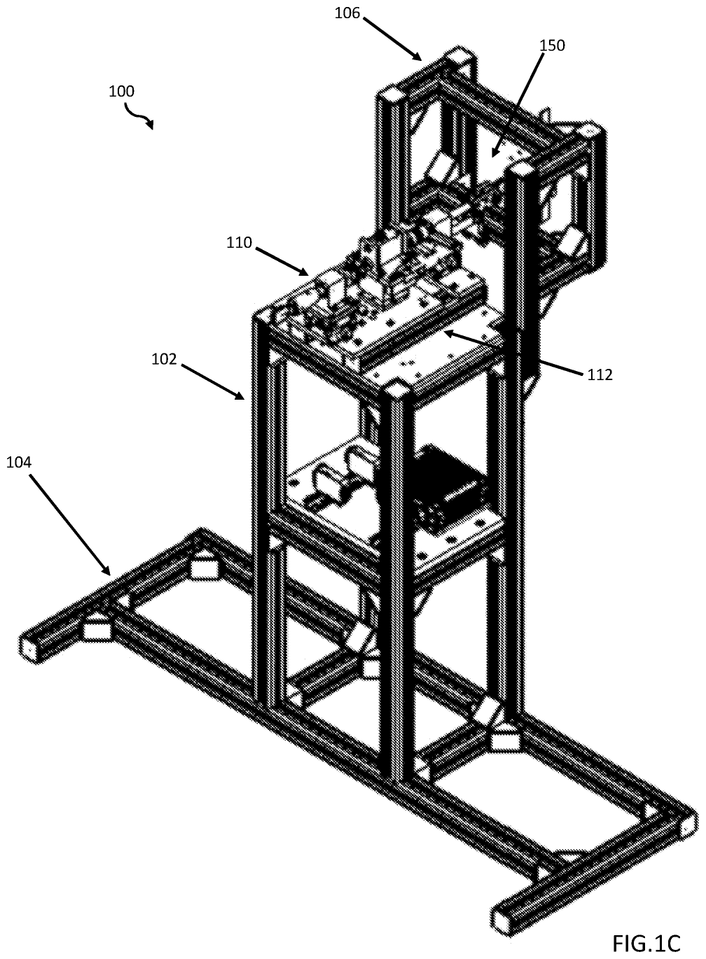

FIG. 2A and FIG. 2B show examples of keys, a lock cylinder and a lock panel which may be used in embodiments of the present invention.

FIG. 2A shows keys and a lock cylinder. The keys 210 have key bow portion 212 and key blade portion 214. The lock cylinder 220 has a cylinder portion 222 with a key hole 224 and a housing portion 226. An actuator 228 is coupled to the cylinder portion 222. In order to lock or unlock a lock, the key blade portion 214 is inserted into the key hole 224 of the lock cylinder 220 and the key bow portion 214 is griped by a user to turn the cylinder portion 222 relative to the housing portion 226. This causes the actuator 228 to move relative to the housing portion 226 and thus actuate the bolt of a lock.

FIG. 2B shows the lock panel. The lock panel 250 has an opening 252 which receives the lock cylinder 222. A bolt 254 is actuated by movement of the actuator 228 when a key 210 is turned in the lock cylinder 220. FIG. 2B also shows a lock plate 256 which is mounted onto a door frame to receive the bolt 254 of the lock panel 250. The lock plate 256 is not generally used in embodiments of the present invention.

FIG. 3A shows a simulated key portion of a motion motor test system according to an embodiment of the present invention. The simulated key portion 122 has a cylindrical body portion 310 and a key bow portion 320 which has a first flat portion 322 and a second flat portion 324. The first flat portion 322 and the second flat portion 324 are arranged to form opposing surfaces of the key bow portion 320. The size and thickness of the key bow portion is selected to correspond to the size of thickness of the bow portion of a key. A miniature load cell is arranged between the first flat portion 322 and the second flat portion 324 as shown in FIG. 3B.

FIG. 3B shows the arrangement of a load cell within the key bow portion of the simulated key portion shown in FIG. 3A. The first flat portion 322 is omitted from the simulated key portion 122 shown FIG. 3B. As shown in FIG. 3B, a miniature load cell 330 is arranged between the first flat portion 322 and the second flat portion 324. The miniature load cell 330 measures the pinch force exerted by the thumb and index finger of the subject when carrying out the turning key test.

FIG. 3C shows an example of a miniature load cell used in embodiments of the present invention. As shown in FIG. 3C, the miniature load cell has a body portion 332 with a sensing element 334. The sensing element 334 measures a force applied to the miniature load cell 330. A cable 336 extends from the body portion 332 and the miniature load cell 330 generates a signal on the cable 336 indicating the force applied to the miniature load cell 330.

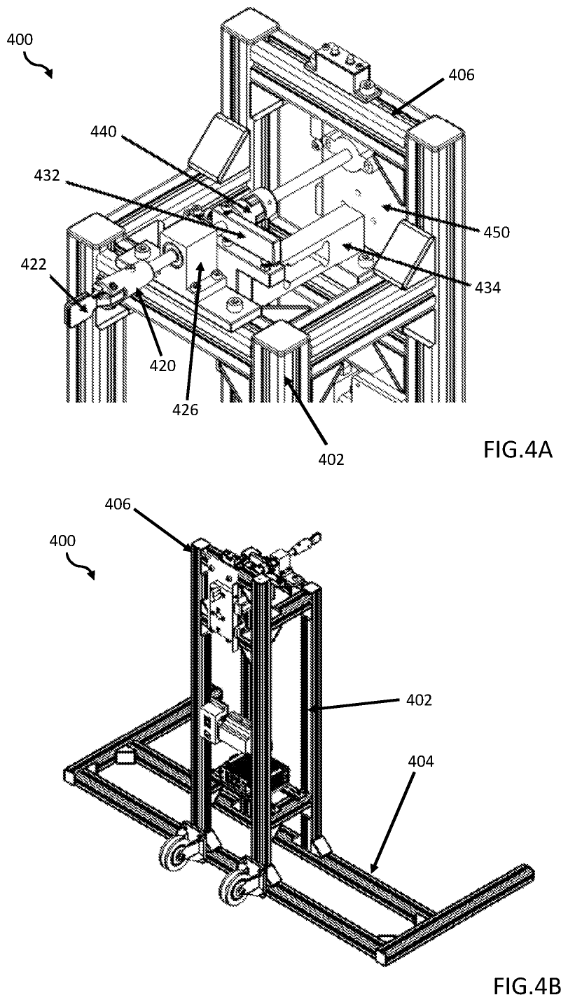

FIG. 4A and FIG. 4B show a motion motor test system according to an embodiment of the present invention. The motion motor test system 400 shown in FIG. 4A and FIG. 4B, differs from the motion motor test system 100 shown in FIG. 1A to FIG. 1C in that it does not utilize the torque sensor, instead, it only measures both pinch and turning force.

As shown in FIG. 4A, the motion motor test system 400 comprises a rotatable assembly. In this embodiment, the rotatable assembly is supported by a bearing block 426 which is mounted on a main frame portion 402. The rotatable assembly comprises a simulated key portion 420 which has a key bow portion 422. The simulated key portion 420 is rotatable around a rotational axis and the bearing block 426 allows the simulated key portion 420 and other parts of the rotatable assembly to be rotated around the rotational axis. An arm portion 432 is mounted on the rotatable assembly. The arm portion 432 extends radially from the rotational axis. A load cells 434 is mounted in an offset position from the rotatable axis. A coupling portion 440 grips a key which is inserted into a lock cylinder of a lock panel 450. The lock panel 450 is supported by a lock panel support portion 406 if the frame of the motion motor test system 400.

As shown in FIG. 4B, the motion motor test system 400 comprises a frame which comprises a main frame portion 402, frame support portions 404 and a lock panel support portion 406. The frame support portions 404 form a base in contact with the floor. The main frame portion 402 extends upwards from the frame support portions 404 and supports the bearing block 426 which in turn supports the rotatable assembly. The lock panel support portion 406 is coupled to the main frame portion 402 and supports the lock panel 450 such that a key hole of the lock panel is located on the rotational axis of the rotational assembly.

The main frame portion may be arranged such that the simulated key portion 420 is positioned at a height of 97 cm to simulate to the common door key height used in flats and buildings.

The motion motor test system may be used to collect data for storage in a database of kinematics and kinetic data collected for upper and lower body tasks. These tasks are either selected from a standardized clinical assessment tool or are representative of daily functional activities such as reaching to grasp an object, turning a key in a lock and walking. The large sample size in the database will be able to capture variations of normal movement patterns, which could provide sufficient data for data-driven healthcare and rehabilitation services and building machine learning models.

The data collected by the motion motor test system can be used and correlated with kinematic data from a motion capture system to enrich the information in the movement database. After data had been correlated, the equipment can then be used in the community setting and collected data from patients. The collected data will be analyzed by using the database as the baseline, to inform the therapists on how the patient movement compared to a normal person movement and what the recovery status.

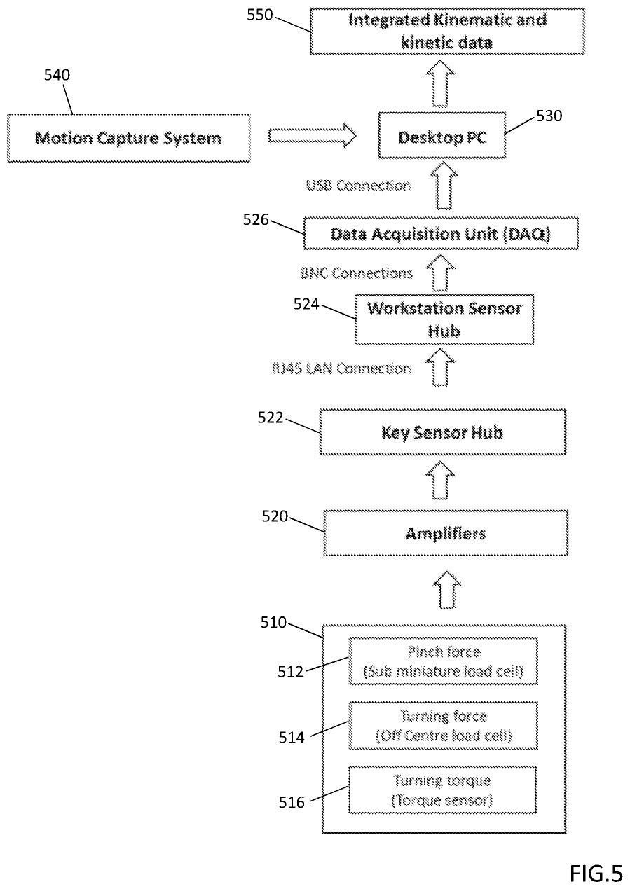

FIG. 5 is a block diagram showing a system for correlating kinetic data captured from a motion motor test system according to an embodiment of the present invention with kinematic data captured by a motion capture system. As shown in FIG. 5, the motion motor test system 510 comprises a pinch force sensor 512, a turning force sensor 514 and a torque sensor 516. The pinch force sensor 512 may correspond to the load cell arranged within the key bow portion of the simulated key portion as described above with reference to FIG. 3A to FIG. 3C. The turning force sensor 514 may correspond to the load cell 134 described above with reference to FIG. 1A or the load cell 434 described above with reference to FIG. 4A. The torque sensor 516 may correspond to the torque sensor 130 described above with reference to FIG. 1A.

The pinch force sensor 512, the turning force sensor 514 and the torque sensor 516 are coupled to amplifiers 520. The amplifiers 520 are coupled to a key sensor hub 522. The key sensor hub 522 is connected to a workstation sensor hub 524 by a local area network (LAN) connection. The workstation sensor hub 524 is connected to a data acquisition unit (DAQ) 526 by Bayonet Neill-Concelman (BNC) connections. The data acquisition unit 526 generates synchronized sensor data using the signals sensed from the sensors.

The output from the data acquisition unit 526 is connected to a desktop computer 530. The desktop computer 530 analyses the synchronized sensor data. This analysis may be based on motion capture data from a motion capture system 540. The motion capture system captures kinematic data of a subject while they complete motion motor task. This kinematic data is synchronized with kinetic data (such as pressure data from the load cells and timing data from the touch sensors) to generate integrated kinematic and kinetic data 550. Once this synchronization is completed, the data from the touch sensors can be used as a surrogate for temporal parameter measurements without the need for motion capture system.

After data has been correlated, the equipment can then be used in a community setting to collected data from patients without the need for a motion capture system. The collected data can be analyzed to inform therapists on how the patient movement compared to a normal person movement and what the recovery status.

Whilst the foregoing description has described exemplary embodiments, it will be understood by those skilled in the art that many variations of the embodiments can be made within the scope and spirit of the present invention.

1. A motion motor test system comprising:
a simulated key portion which is rotatable around a rotational axis;
a rotational resistive load mechanically coupled to the simulated key portion; and
a sensor subsystem coupled to the simulated key portion configured to measure force and/or torque applied to the simulated key portion.

2. The motion motor test system according to claim 1, wherein the simulated key portion comprises a key bow and the sensor subsystem comprises a load cell provided in the key bow configured to measure a pinch force applied to the key bow.

3. The motion motor test system according to claim 2, wherein the key bow comprises two flat portions and the load cell is provided between the two flat portions.

4. The motion motor test system according to claim 1, further comprising: an arm portion mechanically coupled to the key portion and extending radially from the rotational axis, and wherein the sensor subsystem comprises a first load cell mounted in a position offset from the rotational axis and configured to measure a force applied by the arm portion.

5. The motion motor test system according to claim 4, wherein the first load cell is mounted in a rotational position at which a key turning action is completed.

6. The motion motor test system according to claim 4, further comprising a second load cell mounted in a position offset from the rotational axis and configured to measure a force applied by the arm portion.

7. The motion motor test system according to claim 6, wherein the first load cell is mounted in a rotational position at which a key turning action is completed in a first rotational direction and the second cell is mounted in a rotational position at which a key turning action is completed in a second rotational direction opposite from the first rotational direction.

8. The motion motor test system according to claim 1, wherein the sensor subsystem comprises a torque sensor mechanically coupled to the simulated key portion and configured to measure a torque around the rotational axis applied to the simulated key portion.

9. The motion motor test system according to claim 1, wherein the rotational resistive load is a lock and a key.

10. The motion motor test system according to claim 1, further comprising a data acquisition unit configured to collect signals from the sensor subsystem and generate kinetic data for the subject based on the signals from the sensor subsystem.

11. The motion motor test system according to claim 10, further comprising a data synchronization unit configured to synchronize the kinetic data for the subject with kinematic data for the subject captured from a motion capture system.
